(19) United States
(12) Patent Application Publication
(10) Pub. No.: US 2022/0233418 A1
(43) Pub. Date: Jul. 28, 2022

(54) SKINCARE FORMULATIONS WITH POLYGONAL PRISMATIC PLATELET UV FILTERS

(71) Applicant: ENTEKNO ENDUSTRIYEL TEKNOLOJIK VE NANO MALZEMELER SANAYI VE TICARET A.S., Eskisehir (TR)

(72) Inventors: Mehmet Tumerkan KESIM, Eskisehir (TR); Zuhal SAPAN, Eskisehir (TR)

(73) Assignee: ENTEKNO ENDUSTRIYEL TEKNOLOJIK VE NANO MALZEMELER SANAYI VE TICARET A.S., Eskisehir (TR)

(21) Appl. No.: 17/615,161

(22) PCT Filed: May 31, 2019
(86) PCT No.: PCT/TR2019/050415

§ 371 Date: Nov. 30, 2021

(51) Int. Cl. A61K 8/27; A61Q 1/06; A61Q 17/00; A61Q 17/04; A61Q 19/00; A61Q 19/10; A61Q 5/06

(52) U.S. Cl. CPC A61K 8/27; A61Q 1/06; A61Q 17/005; A61Q 17/04; A61K 2800/41; A61Q 19/001; A61Q 19/10; A61Q 5/06; A61K 2800/30; A61Q 19/00

(57) ABSTRACT

A skincare formulation including a prismatic UV filter and polygonal Zinc oxide prismatic platelets composed of primary nanoparticles as an active substance, wherein the skincare formulation is preservative-free or includes one or more preservatives in a total concentration of 2% (w/w) or less with respect to a total weight of the skincare formulation.

### CROSS REFERENCE TO THE RELATED APPLICATIONS

This application is the national stage entry of International Application No. PCT/TR2019/050415, filed on May 31, 2019, the entire contents of which are incorporated herein by reference.

### TECHNICAL FIELD

The present invention relates to skincare formulations with prismatic platelet UV filters. In particular, the present invention relates to topically applicable skincare formulations comprising prismatic platelet UV filters.

### BACKGROUND

Physical UV filters included as an active substance in commercially available skincare or cosmetic formulations, are in the form of powders with spherical or isotropical geometries. Skincare formulations with spherical active substance (in particular when the active substance is not subjected to a chemical surface modification) have several performance limitations as listed below
only a SPF (sun protection factor) value within an usual range between 0.8 and 1.1 is available, with an active substance concentration of 1 wt. % with respect to the total weight of the formulation because of the low efficacy as a result of poor surface coverage;
considering that the maximum available or allowable concentration of ZnO is 25 wt. % with respect to the total weight of the formulation, it is impossible to achieve a SPF50 product (a formulation which has a SPF value of 50) in which only ZnO is used as active substance;
such physical UV filters as active substance are insufficient in skin protection under blue light (i.e. at blue light region of the electromagnetic spectrum), and this shortcoming is only overcome using an additional, chemical UV filter;
skin protection is still limited even when chemical UV filters are employed along with physical UV filters as active substance in the formulations;
the formulations include high amounts of active substance;
formulations which include such physical UV filters as active substance, cause an undesired optical appearance which is called “whitening effect”;
formulations which include such physical UV filters as active substance require the addition of high amounts of preservatives for achieving an acceptable shelf live, which may be hazardous to the health, in particular when such formulations are applied onto the skin in high amounts and/or with high frequency.

Accordingly, the skincare and cosmetic formulations require improvement to overcome the above listed shortcomings.

### SUMMARY

Primary object of the present invention is to overcome the abovementioned shortcomings of the prior art.

Another object of the present invention is provision of a formulation which provides an enhanced extent of sun protection.

A further object of the present invention is provision of a formulation which provides an extent of sun protection comparable with those available in the market, even with decreased concentrations of active substance therein; or even when a physical UV filter is used as active substance.

An even further object of the present invention is provision of a formulation which has a highly or completely decreased health hazard when such formulations are applied onto the skin in high amounts and/or with high frequency, when compared to those available in the market.

An even further object of the present invention is provision of a formulation which does not cause whitening effect.

An even further object of the present invention is provision of a formulation which has optical transparency under visible light, when in the absence of color pigments.

These objectives are achieved by the present invention, which proposes a skincare formulation comprising an active substance in the form of polygonal Zinc oxide-based prismatic platelets composed of primary nanoparticles.

### BRIEF DESCRIPTION OF THE DRAWINGS

The figures, whose brief explanation is herewith provided, are solely intended for providing a better understanding of the present invention and are as such not intended to define the scope of protection or the context in which said scope is to be interpreted in the absence of the description.

FIG. 1 shows comparison of UV protection performances of formulations according to the present invention, in terms of SPF values available with different weight percentages of ZnO-based physical UV filters, with respect to the total weight of each formulation sample.

FIG. 2 shows a shear rate vs. viscosity curve, representing the rheological behavior of a formulation sample according to the present invention.

FIGS. 3A to 3D show the antimicrobial activity of the platelets in various exemplary formulations according to the present invention.

### DETAILED DESCRIPTION OF THE EMBODIMENTS

Referring now the figures outlined before, the present invention proposes a skincare formulation comprising an active substance in the form of polygonal Zinc oxide prismatic platelets composed of primary nanoparticles. The formulation can preferably be in the form of crème, liquid or spray; and preferably provided as a cosmetic sunscreen or an intertrigo remedy formulation.

Said platelets as active substance, can be obtained in accordance with any version of the method described in the international publication number WO 2019/054954 A1. Accordingly, said platelets can include any embodiment of the platelets described in the international publication number WO 2019/054954 A1. The commercial product with the brand name MicNo is an example to such platelets. The platelets can further be coated or surface modified, e.g. by being subjected to chemical treatment prior to introduction into the formulation.

The platelets can be also surface treated. The platelets can be coated with organic and/or inorganic compounds to enhance dispersability, UV protection efficacy and chemical stability of powders.

The organic coatings can be silane or silicon compounds, fatty acids, fatty alcohols, waxes or a combination thereof. Inorganics such as silica, alumina, titania are some examples of surface modifying agents. Organic/inorganic hybrid coatings can also be applied to platelets. The application of unmodified and surface modified platelets are given in example formulations.

The active substance can include polygonic Zinc oxide prismatic platelets composed of primary nanoparticles, said platelets having a size in micron-scale and a median specific surface area of more than 25 square meters per gram, and even 30 square meters per gram, which in turn is advantageous in terms of surface covering capability.

The platelets can include a plurality of primary nanoparticles in a height direction substantially perpendicular to a couple of substantially parallel prism base surfaces having an area greater than any prism side surfaces thereof.

The platelets can include Silver as a further metal in elementary form, or in form of one or more oxides and/or one or more salts thereof. The platelets can alternatively or further include one or more of Aluminium, Gallium and Indium, as a further metal in elementary form, or in form of one or more oxides and/or one or more salts thereof. The platelets can alternatively or further include one or both of Iron and Cobalt as a further metal in their elementary form, or in form of one or more oxides and/or one or more salts thereof.

Since the surface coating capability of the platelets are higher than that of the physical UV filters in the form of spheres (spherical UV filters), the SPF extent available with formulations including the platelets as active substance is up to twice of that available with the spherical UV filters. With formulations according to the present invention, an SPF value within a range between 1.5 and 2.0 is available, with an active substance concentration of 1 wt. % with respect to the total weight of the formulation.

With the maximum allowable or available concentration of ZnO of 25 wt. % with respect to the total weight of the formulation, it is rendered possible to achieve a SPF50 product in which ZnO (ZnO-based platelets) is used as active substance, even without requiring the introduction of any further active substance.

Sun protection under blue light is available with the formulations according to the present invention, even without requiring the introduction of any further active substance.

The sun protection available with the formulations according to the present invention reaches to an extent of 150% or even higher when compared to those including spherical UV filters, especially in the case where the formulation comprises a further active substance in the form of a chemical UV filter.

It is witnessed that the platelets in the formulation show a preservative behavior, dramatically decreasing or even eliminating the necessity of introduction of further preservatives to achieve acceptable or allowable shelf lives.

The platelets can have a thickness providing a light transmittance of 30% or more at a wavelength of 600 nanometers; which thickness being well achievable, as with the method described in said publication. Such low thickness allows transparency, and eliminates the whitening effect when the formulation is applied on the skin. Thus, a substantially transparent formulation is available, in particular when no additional pigments are introduced into the formulation in a concentration to alter the optical appearance of the formulation when in use towards a greater extent of opacity.

For better utilising shape-related advantages of the active substance, the formulation can be designed to render the rheology of the formulation to allow an enhanced-term suspension of the active substance in a uniform fashion. Said shape-related advantages are in particular in the terms of the above-mentioned surface coating capability and/or transparency, without compromising sun protection and without necessitating an increased concentration of the active substance. Said design can correspond to a method for preparation of the formulation, which includes a sequence for mixing various components of the formulation.

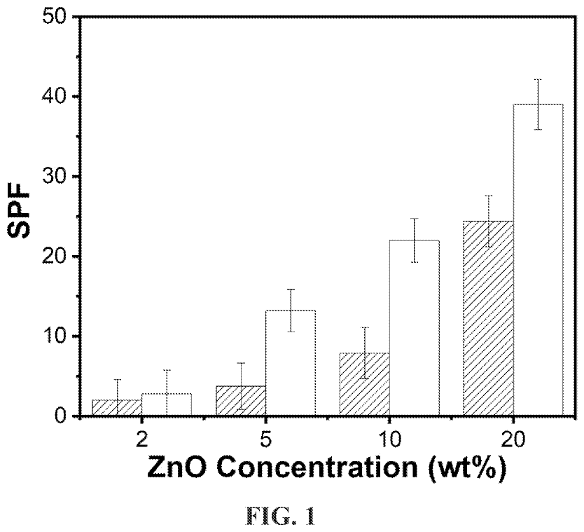

FIG. 1 shows UV protection performance of formulations according to the present invention, in terms of SPF values available with different weight percentages of ZnO-based active substances (here: physical UV filters), with respect to the total weight of each formulation sample. The SPF values shown in FIG. 1 are the results of in-vivo tests. In FIG. 1, ZnO-based platelets (white bars) as active substance according to the present invention, are compared with commercially available ZnO nanospheres (shaded bars) as active substance. It is thus evident that SPF values available with platelets are higher than those available with commercially available ZnO nanospheres at each concentration. SPF value vs. weight percentage of the platelets within the formulation, can be used as a figure of merit for an efficient UVB protection performance. High spreadability and surface coverage ability of the platelets in the formulations according to the present invention, can be responsible for the enhanced UV protection efficacy compared to that available using commercially available ZnO nanospheres as active substance; thereby allowing formulators to use less amount of the physical UV filter to achieve a desired extent of UV protection performance.

An SPF value of around 3 is available with a platelet concentration of only around 2 wt. % with respect to the total weight of the formulation, whereas the SPF value available with ZnO spherical nanopowders is only lower, i.e. around 2 at same concentrations in comparative formulations. Thus, in a preferred embodiment, the formulation includes said platelets as active substance, in a concentration within the range between 1.4 wt. % and 2.6 wt. %, more preferably within the range between 1.8 wt. % and 2.2 wt. %, with respect to the total weight of the formulation. More preferably, such formulation does not include any further physical or chemical active substance for sun protection.

In another preferred embodiment, the formulation includes said platelets as active substance, in a concentration of up to 25 wt. %, with respect to the total weight of the formulation. Thus, a SPF value of 50 is achievable with the formulation, without exceeding the current ZnO limits allowable in the European Union (EU).

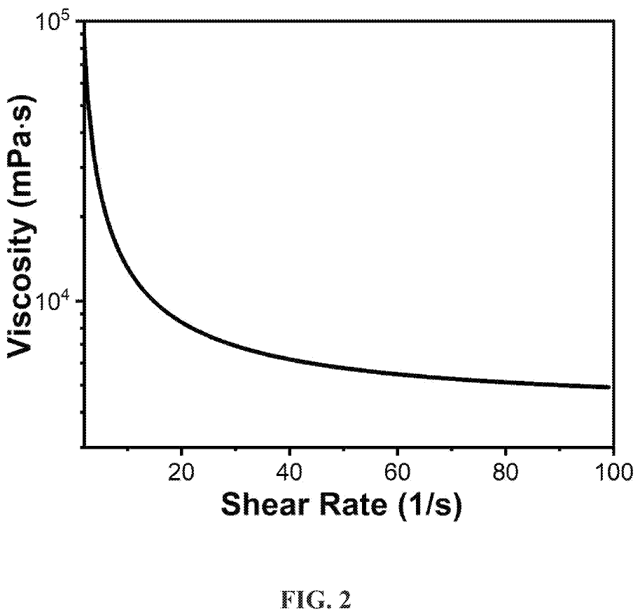

FIG. 2 shows a shear rate vs. viscosity curve, representing the rheological behavior of a formulation sample (given in the Example 1 below) according to the present invention. A characteristic shear thinning rheology can be identified readily with kinematic viscosity values between 10³ and 10⁶ mPa·s, and a dynamic viscosity values between 10² and 10⁴ mPa·s, respectively. This corresponds to thixotropic behavior when the formulation is applied onto human skin, thus a good spreadability.

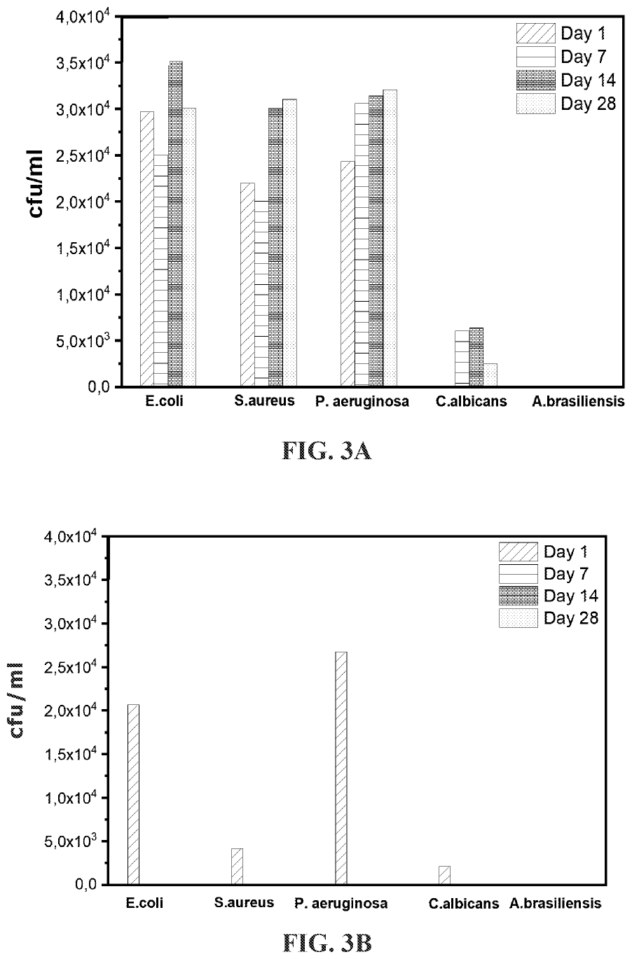

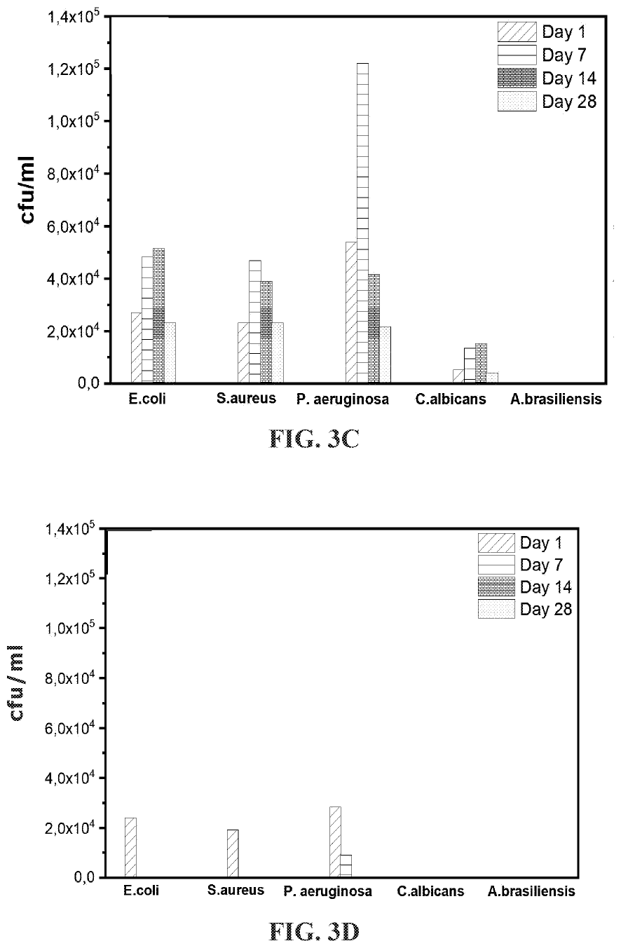

FIGS. 3A to 3D show the antimicrobial activity of the platelets in various exemplary formulations according to the present invention, said formulations also having sun protection because of the presence of said platelets therein. Microbial activity in base creams without such platelets are also given for comparison. It was found that microorganism growth in such base creams, even at day 1, except with A. Brasiliensis. On the other hand, the growth of C. Albicans is suppressed both in the Example I and Example II below, which correspond to formulations named W/O-1 and W/O-2, respectively, and which are prepared as base creams without preservatives. Moreover, S. Aureus growth in the Example II (formulation named W/O-2) is lower in comparison with to its amount in the cream base. The presence of platelets inhibit the growth of certain microorganisms, but it is also witnessed that the platelets further have a lethal effect on most of the bacteria at day 7, with complete preservation at days 14 and 28. Thus, the formulations in the present invention exhibit a substantial self-preservation behavior, thanks to multi-functionality of the platelets acting both as active substance in the terms of UV protection, and as preservation agent.

In an embodiment according to the present invention, the formulation includes a chemical UV filter (e.g. an organic filter) as a further active substance, along with the platelets as active substance. The chemical UV filter as the further active substance can be included into the formulation in the form of a dispersion. Such dispersion can be a base cream, and can be commercially available. When mixed with a chemical UV filter, the platelets as active substance add to the SPF value synergistically, and an “SPF boosting effect” can be calculated by subtracting the SPF value of the chemical UV filter (e.g. of a dispersion or base cream which includes the chemical UV filter) from the final SPF value of the formulation.

For instance, Table 1.1 shows physical UV filter types and their concentrations in four different comparative formulations, namely #1 to #4. #1 and #3 correspond to comparative formulations in which the physical UV filter is commercially available transparent ZnO based nanospheres (CTN-ZnO), with concentrations of 10 wt. % and 15 wt. % with regard to the total weight of the compositions, respectively. #2 and #4 correspond to formulations according to the present invention in which the physical UV filter is ZnO-based platelets with concentrations of 10 wt. % and 15 wt. % with regard to the total weight of the compositions, respectively.

All of said formulations #1 to #4 are prepared by introducing respective types and amounts of physical active substances into a chemical UV filter provided in the form of a base cream. In the comparative samples here, the base cream includes a dispersion of an encapsulated organic sunscreen ingredient which is a commercially available product under the trade name “SunCat DE”, at a concentration of 5 wt. % to achieve a SPF value of 25 with the base cream. Table 1.2 shows respective critical wavelength, transparency percentage at 400 nm, final SPF value, and SPF boosting effect with each of the formulations #1 to #4. Colipa 2006 International SPF and 2011 UVAPF guidelines were followed for in-vitro measurements.

As seen on SPF 25 provided by 5 wt. % of SunCat DE combined with 10 wt. % and 15 wt. % of Platelets (MicNo) provide a SPF boosting effect of 35 and 50, respectively. These values correspond to SPF boosting values of 3.5 and 3.33 per each wt. % increment of the platelets, respectively. Taking into account that the platelets alone would provide an SPF value of about 1.5 per each wt. % increment thereof, the results show that the combination of the platelets with an organic UV filter (which is merely exemplified over SunCat DE) has a clear synergistic effect.

This synergistic effect is much higher than that available with the combination of CTN-ZnO and SunCat DE, when compared with the results shown for the samples #1 and #3; in particular when the formulation includes less than 15 wt. % of the platelets combined with an organic UV filter as seen with the sample #2.

TABLE 1.1
Physical UV filter concentrations in exemplary
formulation samples, which further
include a chemical UV filter as a further active substance.

| | | Concentration of the physical |
|---|---|---|
| | Physical | UV filter in the formulation |
| | UV | (in wt. % with respect to the total |
| # | filter | weight of the formulation) |
| 1 | CTN-ZnO | 10 |
| 2 | Platelets | 10 |
| 3 | CTN-ZnO | 15 |
| 4 | Platelets | 15 |

TABLE 1.2
Several characteristics of the exemplary formulation
samples given in the Table 1.1.

| | Critical | | Transparency | Base | | SPF |
|---|---|---|---|---|---|---|
| | Wavelength | | (%, at | Cream | Final | Boosting |
| # | (nm) | UVA/UVB | 440 nm) | SPF | SPF | Effect |
| 1 | 376 | 0.64 | 71 | 25 | 40 | 15 |
| 2 | 375 | 0.60 | 72 | 25 | 60 | 35 |
| 3 | 377 | 0.75 | 58 | 25 | 70 | 45 |
| 4 | 375 | 0.65 | 65 | 25 | 75 | 50 |

It is evident from Table 1.1 and Table 1.2 that each wt. % platelets concentration increase provide more than 3 SPF to the formulation: in #2 and #4, SPF boosting effects of 35 and 50 are achieved by 10 wt. % (thus +3.5 SPF per wt. %) and 15 wt. % (thus +3.33 SPF per wt. %) of the platelets in the formulations, respectively. Yet, the SPF boosting effect available using CTN-ZnO is only 15/10=1.5 and 45/15=3 for #1 and #3, respectively. Hence, in the formulation according to the present embodiment of the invention, the platelets as active substance and the chemical UV filter as further active substance being mixed, result in a synergistic effect in the terms of SPF boosting effect. Accordingly, in a preferred embodiment according to the present invention, the formulation includes a chemical UV filter, more preferably an organic UV filter, even more preferably an encapsulated organic sunscreen dispersion, as a further active substance. Table 1.2 also shows that the transparency values at 440 nm which is around the middle of the blue light zone of the electromagnetic spectrum, are maintained when the active substance is platelets instead of CTN-ZnO.

The formulations according to the present invention can further include one or more preservatives which are suitable for tuning (e.g. prolonging) shelf life of cosmetic products. Any skincare formulation according to the present invention is preservative-free or includes one or more preservatives in a total concentration of 2% (w/w) or less with respect to the total weight of the formulation. The platelets can have a total concentration preferably within the range between 1 wt. % and 25 wt. %, more preferably within the range between 10 wt. % and 20 wt. %, with respect to the total weight of the formulation.

ZnO is known to be an attractive antimicrobial agent especially when presented in nano form due to their high specific surface area. Broad spectrum antimicrobial activity of the platelets for being employed in the formulations according to the present invention, were previously demonstrated by Demirel et al., (Journal of Drug Delivery Science and Technology, Volume 47, October 2018, Pages 309-321).

In a possible embodiment according to the present invention, the formulation is a sunscreen. In another possible embodiment according to the present invention, the formulation is a rash crème.

In another possible embodiment according to the present invention, the formulation is a moisturizing crème. In another possible embodiment according to the present invention, the formulation is a hair gel. In another possible embodiment according to the present invention, the formulation is a lipstick. In another possible embodiment according to the present invention, the formulation is a lip moisturizing stick. In another possible embodiment according to the present invention, the formulation is a lip balm. In another possible embodiment according to the present invention, the formulation is a hand sanitizer.

### Exemplary Experiments

The exemplary experiments explained below are provided solely for better understanding of the present invention. Examples I to VI do not include any preservative (i.e. any preservative known for being suitable for enhancing shelf live by suppressing bacterial growth in skincare formulations). Examples VII to XI include preservatives for enhancing the shelf life of respective formulations. In each example, an exemplary composition of the respective formulation with functions and weight percentages of components constituting the composition is given, with respect to the total weight of each respective composition. In each example, the column named “phase” corresponds to materials or mixtures which are preferably separately prepared and only then mixed together with the other phases; to achieve an enhanced uniformity and suspension stability in the respective formulations.

### Example I (W/O-1)

Example I introduces an exemplary composition (coded as W/O-1), which includes a water-in-oil emulsion. Phase A can be an oil-based mixture which can include one or more of emollients, dispersants, emulsifiers and antioxidants; whereas the Phase B can be a water-based mixture which can include demineralized water, one or more of emollients and thickeners. Phases A and B can be separately prepared by mixing their respective ingredients, and melted (e.g. at 80° C., in a water bath). For preparation of the Phase B, xanthan gum can be dissolved in glycerol and their mixture is added into water. The Phase B can be then slowly added into the Phase A under stirring, preferably using a paddle stirrer. The resulting emulsion which includes the mixture of Phase A and Phase B can be preferably further stirred, e.g. for 5 minutes. Phase C can include ZnO-based platelets which correspond to an active substance (physical UV filter), e.g. those commercially available under the brand name “MicNo”. The Phase C can be slowly added into the emulsion to obtain a formulation according to the present invention, in the form of a suspension. The suspension is preferably subjected to homogenization in order to enhance the uniformity of the formulation: and the suspension can be then subjected to stirring preferably using a propeller stirrer, e.g. for 5 minutes. The resulting formulation can be then cooled down to room temperature. “INCI Name” corresponds to the name of a component according to the “international nomenclature of cosmetic ingredients”.

An exemplary composition of the embodiment according to the Example I is presented in the Table 2.1.

### Example II (W/O-2)

Example II introduces an exemplary composition (coded as W/O-2), which includes a water-in-oil emulsion. Phase A can be an oil-based mixture which can include one or more of emollients, dispersants and emulsifiers; whereas the Phase B can be a water-based mixture which can include demineralized water, one or more of emollients and thickeners. Phase C includes ZnO-based platelets which corresponds to an active substance (physical UV filter), e.g. those commercially available under the brand name MicNo.

Phases A and B can be separately prepared by mixing their respective components, and melted (e.g. at 70° C., in a water bath). The Phase B can be then slowly added into the Phase A under stirring, preferably using a paddle stirrer. The resulting emulsion which includes the mixture of Phase A and Phase B can be preferably further stirred, e.g. for 5 minutes. The Phase C can be slowly added into the emulsion, to obtain a suspension. The suspension is preferably subjected to homogenization in order to enhance the uniformity of the suspension. The Phase D, which can be a further oil-based mixture including one or more of emollients and antioxidants, can be then added into the suspension, to obtain a further formulation according to the present invention. Said addition can be preferably made when the temperature of the suspension drops down to a value below 45° C., more preferably to a value of no more than 40° C. The suspension which also includes the ingredients from Phase D, can be then subjected to stirring preferably using a propeller stirrer, e.g. for 5 minutes. The resulting formulation can be then cooled down to room temperature.

An exemplary composition of the embodiment according to the Example II is presented in the Table 2.2.

### Example III

Example III introduces an exemplary composition (coded as W/Si), which includes a water-in-Silicon oil emulsion. Phase A can be an oil-based mixture which can include one or more of emollients and emulsifiers; whereas the Phase B can be a water-based mixture which can include demineralized water, one or more of humectants and thickeners. Phase C can be a mixture which can include one or more of film forming agents and emollients and/or solvents. Phase D can include ZnO-based platelets which correspond to an active substance (physical UV filter), e.g. those commercially available under the brand name “MicNo”.

Phases A and B can be separately prepared by mixing their respective ingredients. For preparation of Phase A, its components can be mixed and heated e.g. to around 80° C. For preparing the Phase B, the thickener (e.g. xanthan gum) can be added into a humectant (e.g. glycerin) under stirring; and the resulting mixture can be further introduced into water, and then this aqueous mixture can be heated, e.g. to around 70° C. The Phase B can be then added into the Phase A, and then the resulting emulsion which includes the mixture of the contents of the Phase A and Phase B can be preferably further stirred, e.g. for 3 minutes. For preparing the Phase C, its components can be mixed and melted. The Phase C can be slowly added into the emulsion. Then, the Phase D can be slowly added into the emulsion containing the components of Phase A, Phase B and Phase C to obtain a formulation according to the present invention, in the form of a suspension. The suspension is preferably subjected to homogenization in order to enhance the uniformity of the suspension. The resulting formulation can be then cooled down to room temperature.

An exemplary composition of the embodiment according to the Example III is presented in the Table 2.3.

### Example IV

The formulation in the Example IV (coded as TiO₂—ZnO mix), is a slightly modified version of the formulation given in Example II. ZnO-based platelets as described above as active substance, and TiO₂ as a further active substance (as a further physical UV filter), are used together in this formulation, mixture of which being exemplified as the Phase C. In the formulation, the TiO₂ concentration can be up to 8 wt. %, whereas the platelets concentration can range between 12 wt. % and 50 wt. % with respect to the total weight of the formulation. The total amount of the platelets and TiO₂ can add up to 20 wt. %, which is identical to the active substance concentration in the formulation shown in the Example II.

Here, the Phase A and Phase B can be prepared separately by mixing their respective components, and by melting the respective mixtures, e.g. at 70° C. and in a water bath. The Phase B can be then slowly added into the Phase A under stirring, preferably using a paddle stirrer. The resulting emulsion which includes the mixture of Phase A and Phase B can be preferably further stirred, e.g. for 5 minutes. Phase C can be then slowly added into the emulsion, to obtain a suspension. The suspension is preferably subjected to homogenization in order to enhance the uniformity of the suspension. The Phase D, which can be a further oil-based mixture including one or more of emollients, humectants and antioxidants, can be then added into the suspension, to obtain a further formulation according to the present invention. Said addition can be preferably made when the temperature of the suspension drops down to a value below 45° C., more preferably to a value of no more than 40° C. The suspension which also includes the ingredients from Phase D, can be then subjected to stirring preferably using a propeller stirrer, e.g. for 5 minutes. The resulting formulation can be then cooled down to room temperature.

An exemplary composition of the embodiment according to the Example IV is presented in the Table 2.4.

### Example V

Example V introduces an exemplary formulation which can be used as a rash cream, or an intertrigo treatment composition. Here, the Phase A can be an oil-based mixture which can include one or more of emollients, emulsifiers, thickeners and antioxidants. The Phase B can include water. The Phase C can include an active substance (physical UV filter) in the form of the ZnO-based platelets (commercially available under the brand name MicNo).

Phase A can be prepared by mixing its components, and melted (e.g. at 70° C., in a water bath).

Phase B can be heated (e.g. to 70° C.) and then added into the Phase A and can be stirred, e.g. for 15 minutes.

An exemplary composition of the embodiment according to the Example V is presented in the Table 2.5.

### Example VI

Example VI introduces an exemplary composition (coded as W/O-4), which includes a water-in-oil emulsion. Phase A can be an oil-based mixture which can include one or more of emollients, thickeners, emulsifiers and antioxidants; whereas the Phase B can be a water-based mixture which can include demineralized water, one or more of humectants and thickeners. Phase C can include ZnO-based platelets which corresponds to an active substance (physical UV filter), e.g. those commercially available under the brand name MicNo; which can further include one or more of emollients and dispersing agents.

Phase A can be prepared by mixing its components, and melted (e.g. at 70° C., in a water bath). Phase B can be prepared by dissolving a thickener in a humectant (e.g. xanthan gum in glycerin), and then heated (e.g. to 70° C.) and then added into the Phase A. Phase C can be prepared by homogenization of its components until a uniform dispersion is formed. Then, the Phase C can be introduced into the mixture including the components of the Phase A and Phase B; and the resulting mixture can be subjected to further homogenization, and then can be cooled down to a temperature value below 45° C., more preferably to a value of no more than 40° C.

An exemplary composition of the embodiment according to the Example VI is presented in the Table 2.6.

### Example VII

Example VII introduces an exemplary composition (coded as W/O-5), which includes a water-in-oil emulsion. Phase A can be an oil-based mixture which can include one or more of emollients, emulsifiers, thickeners, the platelets, and moisturizers. Phase B can be another oil-based mixture which can include one or more of film forming agents, emollients, moisturizers, preservatives, a dispersion of an encapsulated organic sunscreen, and fragrances. Phase C can be an aqueous mixture which can include one or more of thickeners, humectants and chelating agents.

Here, the Phase A and Phase C can be prepared separately by mixing their respective components, and by heating the respective mixtures, e.g. to 70° C. in a water bath. Phase C can be subjected to homogenization under stirring at e.g. 3000 rpm. Phase A can be then mixed into the Phase C, and this mixture can be subjected to further homogenization, e.g. for a duration of 20 minutes. The film former as a component of the Phase B can be introduced into the mixture which includes the components of Phase A and Phase C. Then, this mixture comprising the film former can be cooled down, preferably by a rapid cooling, e.g. by subjecting it into thermal contact with ice. When the temperature drops to a value preferably below 40° C. other components of the Phase B except the dispersion of an encapsulated organic sunscreen (if applicable), can be introduced to the cooled down mixture, and then the resulting mixture can be subjected to homogenization, e.g. for a duration of 15 minutes. Then, said dispersion of encapsulated sunscreen can be introduced thereinto, and stirred.

An exemplary composition of the embodiment according to the Example VII is presented in the Table 2.7. Note that if the amount of zinc oxide in the formulation needs to be changed, deionized water content should be adjusted accordingly to achieve a 100 wt % total ingredient amount. Thus, although the amount of water is exemplified as 43.35 wt. %, this amount only corresponds to a complementary amount.

### Example VIII

Example VIII introduces an exemplary composition (coded as O/W-1), which includes an oil-in-water emulsion, which can be used as a sunscreen formulation. Phase A is an oil-based mixture which can include one or more of emollients, emulsifiers and dispersants. Phase B includes ZnO-based platelets which correspond to an active substance (physical UV filter), e.g. those commercially available under the brand name “MicNo”. Phase C can be an aqueous mixture which can include one or more of humectants and thickeners. Phase D can be a blend of preservatives suitable for use in shelf life adjustment of skincare products.

Here, the components of the Phase C except water can be mixed to form a paste, e.g. a smooth paste. Water can be then added onto the paste, e.g. under stirring. Thus, the Phase C can be obtained. Components of the Phase A can be mixed together, e.g. until a uniform mixture is obtained. Thus, the Phase A can be obtained. Then, the Phase B can be added onto the Phase A, e.g. under vigorous stirring. Then the resulting mixture (which includes the components of Phase A and Phase B) can be subjected to homogenization, e.g. for a duration of about 2 minutes. This mixture can be then added into the Phase C, e.g. under stirring. This can be followed by a homogenization, e.g. for a duration of 1 minute per 100 grams of this mixture (which includes the components of Phase A, Phase B and Phase C). This can be followed by addition of the preservative(s) (i.e. Phase D) to obtain a formulation under stirring, and adjustment of the pH of the formulation to a value within the range between 6.5 and 7.5.

An exemplary composition of the embodiment according to the Example VIII is given in the Table 2.8.

### Example IX

Example IX introduces an exemplary composition which includes an oil-in-water emulsion. Phase A is an oil-based mixture which can include one or more of emollients and/or solvents, thickeners, photostabilizers, organic UVB filters, organic UVA filters and dispersants. Phase B includes ZnO-based platelets which correspond to an active substance (physical UV filter), e.g. those commercially available under the brand name “MicNo”. Phase C can be an aqueous mixture which can include one or more of humectants and thickeners. Phase D can include one or more of preservatives suitable for use in shelf life adjustment of skincare products, and film forming agents (film formers).

Here, the components of the Phase C except water can be mixed, and water can be then added to obtain an aqueous dispersion, e.g. under stirring. The aqueous dispersion can be heated, e.g. to a temperature of 75° C., or even of 80° C.

Separately, the components of the Phase A can be mixed to obtain an oil-based mixture. The oil-based mixture can be then heated, for achieving a decreased viscosity (e.g. by melting of its components) and for enhancing uniformity, e.g. to a temperature of 75° C., or even of 80° C. Then, the Phase B can be introduced to the oil-based mixture, to obtain an oil-based dispersion, e.g. under intensive stirring. The oil-based dispersion can be heated again for achieving a decreased viscosity (e.g. by melting of at least some of its components) and for enhancing uniformity, e.g. to a temperature of 75° C., or even of 80° C. Thus oil-based dispersion can be then introduced into the aqueous dispersion for obtaining a formulation according to the present invention, under stirring. The resulting formulation can be subjected to a homogeneity enhancement (homogenization), e.g. for 1 minute per 100 g. of the formulation; and then can be cooled down under stirring. When the temperature of the formulation decreases, e.g. to below 45° C. or to about 40° C., the formulation can be further modified by introducing the components of the Phase D. This can be followed by adjustment of the pH of the formulation to a value within the range between 6.5 and 7.5.

An exemplary composition of the embodiment according to the Example IX is given in the Table 2.9.

### Example X

Example X introduces an exemplary composition which includes a water-in-oil emulsion. Phase A can be an oil-based mixture which can include one or more of emollients and/or carriers, and emulsifiers. Phase B includes ZnO-based platelets which correspond to an active substance (physical UV filter), e.g. those commercially available under the brand name “MicNo”. Phase B can further include one or more of further inorganic UV filters as further active substances, and dispersants. Phase C can be an aqueous mixture which can include one or more of stabilizers and humectants. Phase D can include one or more of preservatives suitable for use in shelf life adjustment of skincare products.

Here, the components of the Phase A and Phase C can be mixed separately in respective vessels and heated e.g. to a temperature of 75° C., or even of 80° C. Then, the components of the Phase B can be introduced into the Phase A, preferably performed by introducing the components one by one, preferably under vigorous stirring and while maintaining the temperature. Phase C can be then slowly added onto the mixture of Phase A and Phase B, preferably under vigorous stirring. The resulting formulation can be subjected to a homogeneity enhancement (homogenization), e.g. for 1 minute per 100 g. of the formulation; and then can be cooled down under stirring. The resulting formulation can be subjected to a homogeneity enhancement (homogenization), e.g. for 1 minute per 100 g. of the formulation; and then can be cooled down under stirring. When the temperature of the formulation decreases, e.g. to below 45° C. or to about 40° C., the formulation can be further modified by introducing the components of the Phase D.

An exemplary composition of the embodiment according to the Example X is presented in the Table 2.10.

### Example XI

Example XI introduces an exemplary composition which includes a water-in-oil emulsion. Phase A can be an oil-based mixture which can include one or more of emollients, emulsifiers and dispersants. Phase B includes ZnO-based platelets which correspond to an active substance (physical UV filter), e.g. those commercially available under the brand name “MicNo”. Phase C can be an aqueous mixture which can include one or more of thickeners and humectants. Phase D can include one or more of pigments for altering the optical character of the formulation. Phase E can include preservatives suitable for use in shelf life adjustment of skincare products.

Here, the components of the Phase C except water can be mixed to form a paste, e.g. a smooth paste. Water can be then added onto the paste, e.g. under stirring; and this mixture can be then subjected to homogenization. Thus, the Phase C can be obtained. Separately, the components of the Phase A can be mixed together. Then, the Phase B can be added onto the Phase A, e.g. under vigorous stirring. Then the resulting formulation (which includes the components of Phase A and Phase B) can be added into the aqueous Phase C under vigorous stirring, and the formulation can be subjected to a further homogenization, e.g. for a duration of 1 minute per 100 grams of the mixture. The formulation can be then further modified by introduction of pre-ground components of the Phase D, and subjected to an even further homogenization for dispersing the pigments. This can be followed by adjustment of the pH of the formulation to a value within the range between 6.5 and 7.5.

An exemplary composition of the embodiment according to the Example XI is presented in the Table 2.11.

### Tables 2.1 to 2.11

TABLE 2.1
Exemplary composition of the embodiment according to the Example I.

| | | Amount | | |
|---|---|---|---|---|
| Ingredient Name | INCI Name | (wt %) | Function | Phase |
| Polyhydroxystearic | Polyhydroxystearic | 1.0 | Dispersant | A |
| acid | acid | | | |
| Shea Butter | Butyrospermum Parkii | 4.0 | Emollient | |
| | (Shea) Butter | | | |
| Dehymuls | PEG-30 | 3.0 | Emulsifier | |
| | Dipolyhydroxystearate | | | |
| Lameform TGI | Polyglyceryl-3 | 4.0 | Emulsifier | |
| | Diisostearate | | | |
| Cetiol AB | C12-15 Alkyl | 10.0 | Emollient | |
| | Benzoate | | | |
| D-Panthenol | Panthenol | 2.0 | Emollient | |
| Aloe Vera | Aloe Barbadesis Leaf | 1.0 | Emollient | |
| | Juice | | | |
| Vitamin E | Tocopheryl Acetate | 1.0 | Antioxidant | |
| Demineralised | Demineralised water | 72.9 | | B |
| water | | | | |
| Glycerin | Glycerin | 4.0 | Humectant | |
| Xanthan Gum | Xanthan Gum | 0.1 | Thickener | |
| Platelets (MicNo) | Zinc Oxide | 20.0 | UV filter | C |

TABLE 2.2
Exemplary composition of the embodiment according o the Example II.

| | | Amount | | |
|---|---|---|---|---|
| Ingredient Name | INCI Name | (wt %) | Function | Phase |
| Olive Oil | Olea Europaea (Olive) | 15.00 | Emollient | A |
| | Fruit Oil | | | |
| Shea Butter | Butyrospermum Parkii | 5.00 | Emollient | |
| | (Shea) Butter | | | |
| Beeswax | Beeswax | 1.00 | Emollient | |
| Deheymuls | PEG-30 | 3.00 | Emulsifier | |
| | Dipolyhydroxystearate | | | |
| Lameform TGI | Polyglyceryl- 3 | 4.00 | Emulsifier | |
| | Diisostearate | | | |
| Polyhydroxystearic | Polyhydroxystearic | 3.00 | Dispersant | |
| acid | acid | | | |
| Xanthan Gum | Xanthan Gum | 0.08 | Thickener | B |
| Glycerin | Glycerin | 1.00 | Humectant | |
| Demineralised | Demineralised water | 41.92 | | |
| water | | | | |
| MicNo | Zinc Oxide | 20.00 | UV filter | C |
| D-Panthenol | Panthenol | 2.00 | Emollient | D |
| Aloe Vera | Aloe barbadensis leaf | 2.00 | Emollient | |
| | juice | | | |
| Vitamin E | Tocopheryl Acetate | 2.00 | Antioxidant | |

TABLE 2.3
Exemplary composition of the embodiment according to
the Example III.

| | | A- | | |
|---|---|---|---|---|
| Ingredient | | mount | | |
| Name | INCI Name | (wt %) | Function | Phase |
| Shea Butter | Butyrospermum Parkii | 2.0 | Emollient | A |
| | (Shea) Butter | | | |
| Dow Corning | Cetyl Diglyceryl | 3.0 | Emulsifier | |
| 5600 | Tris(Trimethyl- | | | |
| | siloxy)silylethyl | | | |
| | Dimethicone | | | |
| Lameform tgi | Polyglyceryl-3 Diisostearate | 4.0 | Emulsifier | |
| Miglyol 812 N | Caprylic/Capric Triglyceride | 5.0 | Emollient | |
| (F) | | | | |
| Miglyol Gel B | Caprylic/Capric Triglyceride | 3.5 | Emollient | |
| | (and) Stearalkonium | | | |
| | Hectorite | | | |
| | (and) Propylene Carbonate | | | |
| Miglyol coco | Coco Caprylate Caprate | 2.0 | Emollient | |
| 810 | | | | |
| Miglyol OE | Oleyl Erucate | 3.0 | Emollient | |
| Xanthan Gum | Xanthan Gum | 0.1 | Thickener | B |
| Glycerin | Glycerin | 4.0 | Humectant | |
| Demineralized | Demineralized water | 52.4 | | |
| water | | | | |
| DOWSIL™ | Trimethylsiloxysilicate | 3.0 | Film | C |
| MQ- | (and) | | former | |
| 1640Flake | Polypropylsilsesquioxane | | | |
| Resin | | | | |
| Cyclo- | Cyclopentasiloxane | 3.0 | Emollient/ | |
| pentasiloxane | | | solvent | |
| MicNo | Zinc Oxide | 15.0 | UV filter | D |

TABLE 2.4
Exemplary composition of the embodiment according to the Example IV.

| Ingredient | | Amount | | |
|---|---|---|---|---|
| Name | INCI Name | (wt %) | Function | Phase |
| Olive Oil | Olea Europaea (Olive) | 15.00 | Emollient | A |
| | Fruit Oil | | | |
| Shea Butter | Butyrospermum | 5.00 | Emollient | |
| | Parkii | | | |
| | (Shea) Butter | | | |
| Beeswax | Beeswax | 1.00 | Emollient | |
| Deheymuls | PEG-30 | 3.00 | Emulsifier | |
| | Dipolyhydroxystearate | | | |
| Lameform tgi | Polyglyceryl-3 | 4.00 | Emulsifier | |
| | Diisostearate | | | |
| Poly- | Polyhydroxystearic | 3.00 | Dispersing | |
| hydroxystearic | acid | | agent | |
| acid | | | | |
| Xanthan Gum | Xanthan Gum | 0.08 | Thickener | B |
| Glycerin | Glycerin | 1.00 | Humectant | |
| Demineralised | Demineralised water | 41.92 | | |
| water | | | | |
| MicNo | Zinc Oxide | 12.00-20.00 | UV filter | C |
| Titanium | Titanium dioxide | 0-8.00 | UV filter | |
| dioxide | | | | |
| D-Panthenol | Panthenol | 2.00 | Emollient | D |
| Aloe Vera | Aloe barbadensis leaf | 2.00 | Humectant | |
| | juice | | | |
| Vitamin E | Tocopheryl Acetate | 2.00 | Antioxidant | |

TABLE 2.5
Exemplary composition of the embodiment according to the Example V.

| Ingredient | | Amount | | |
|---|---|---|---|---|
| Name | INCI Name | (wt %) | Function | Phase |
| Shea butter | Butyrospermum Parkii | 5 | Emollient | A |
| | (Shea) Butter | | | |
| Dehymuls | PEG-30 | 4 | Emulsifier | |
| PGPH | Dipolyhydroxystearate | | | |
| Lanette O | Cetearyl Alcohol | 2 | Thickener | |
| Lameform TGI | Polyglyceryl-3 | 3 | Emulsifier | |
| | Diisostearate | | | |
| Aleo Vera | Aloe barbadensis leaf | 1 | Emollient | |
| | juice | | | |
| E Vitamini | Tocopherol Acetate | 5 | Antioxidant | |
| Olus Oil | Olus Oil | 4 | Emollient | |
| Vaseline | Petrolatum | 5 | Emollient | |
| D-Panthenol | Panthenol | 5 | Emollient | |
| Demineralised | Demineralised water | 40 | | B |
| water | | | | |
| MicNo | Zinc Oxide | 20 | UV filter | C |

TABLE 2.6
Exemplary composition of the embodiment according to the Example VI.

| | | Amount | | |
|---|---|---|---|---|
| Ingredient Name | INCI Name | (wt %) | Function | Phase |
| Jojoba oil | Simmondsia | 10.0 | Emollient | A |
| | Chinensis (Jojoba) | | | |
| | Seed Oil | | | |
| Lanette o | Cetearyl Alcohol | 4.0 | Thickener | |
| Shea Butter | Butyrospermum Parkii | 5.0 | Emollient | |
| | (Shea) Butter | | | |
| Deheymuls | PEG-30 | 3.0 | Emulsifier | |
| | Dipolyhydroxystearate | | | |
| Lameform tgi | Polyglyceryl-3 | 4.0 | Emulsifier | |
| | Diisostearate | | | |
| D-Panthenol | Panthenol | 5.5 | Emollient | |
| Aloe Vera | Aloe barbadensis leaf | 3.0 | Emollient | |
| | juice | | | |
| E Vitamini | Tocopheryl Acetate | 5.0 | Antioxidant | |
| Glycerin | Glycerin | 1.5 | Humectant | B |
| Xanthan Gum | Xanthan Gum | 0.1 | Thickener | |
| Demineralised | Demineralised water | 28.9 | | |
| water | | | | |
| MicNo | Zinc Oxide | 20.0 | UV filter | C |
| Cetoil AB | C12-15 Alkyl | 9.08 | Emollient | |
| | Benzoate | | | |
| Dispersun OL | Polyhydroxystearic | 0 92 | Dispersing | |
| 300 | Acid | | agent | |

TABLE 2.7
Exemplary composition of the embodiment according to the Example VII.

| Ingredient | | Amount | | |
|---|---|---|---|---|
| Name | INCI Name | (wt %) | Function | Phase |
| Emulgade | Glyceryl stearate | 6 | Emulsifier | A |
| SE PF | (and) Ceteareth-20 | | | |
| | (and) Ceteareth-12 | | | |
| | (and) Cetearyl | | | |
| | Alcohol (and) | | | |
| | Cetyl Palmitate | | | |
| Lanette O | Cetearyl Alcohol | 2 | Thickener | |
| Olive Oil | Olea Europaea | | | |
| | (Olive) Fruit Oil | | | |
| Shea Butter | Butyrospermum Parkii | 1.25 | Emollient | |
| | (Shea) Butter | | | |
| SF 1256 | Cyclohexasiloxane (and) | 1 | Emollient | |
| | cyclopentasiloxane | | | |
| MIGLYOL | Caprylic/Capric | 5 | Emollient | |
| 812 | Triglyceride | | | |
| Cetiol AB | C12-15 Alkyl Benzoate | 5 | Emollient | |
| Cetiol CC | Dicaprylyl Carbonate | 1.50 | Emollient | |
| Ceraphyl 368- | Ethylhexyl Palmitate | 0.30 | Emollient | |
| Palmester | | | | |
| 1545 | | | | |
| Ceraphyl 847 | Octyldodecyl | 0.30 | Emollient | |
| | Stearoyl Stearate | | | |
| Dowcorning | Caprylyl Methicone | 0.75 | Emulsifier | |
| EL 7040 | (and) PEG-12 | | | |
| | Dimethicone/PPG-20 | | | |
| | Crosspolymer | | | |
| MicNo | Zinc Oxide | 10 | UV filter | |
| Orchid | Caprylic/Capric | 0.20 | Moisturizer | |
| Complex | Triglyceride (and) | | | |
| | Cymbidium | | | |
| | Grandiflorum (Orchid) | | | |
| | Flower Extract | | | |
| Baysilon M | | 0.60 | Emollient | |
| 100-Oil | | | | |
| M100 | | | | |
| Raphitix A | Sodium Polyacrylate | 0.80 | Film | B |
| 100 | | | former | |
| Aloe Vera | Aloe barbadensis | 0.30 | Emollient | |
| | leaf juice | | | |
| Chamomile | Glycerin (and) | 0.25 | Moistutizer | |
| Extract | Water (and) | | | |
| | Chamomilla Recutita | | | |
| | (Matricaria) | | | |
| | Flower Extract | | | |
| Versatil SL | Aqua (and) Sodium | 2.00 | Preservative | |
| | Levulinate (and) | | | |
| | Potassium Sorbate | | | |
| Unitrenol T | Farnesyl Acetate | 0.60 | Fragrance | |
| 27 | (and) Farnesol | | | |
| | (and) Panthenyl | | | |
| | Triacetate | | | |
| SunCat DE | Aqua (and) | 5 | UV filter | |
| | Benzophenone-3 (and) | | | |
| | Butylene Glycol (and) | | | |
| | Phospholipids (and) | | | |
| | Ethylhexyl | | | |
| | Methoxycinnamate (and) | | | |
| | Butyl | | | |
| | Methoxydibenzoylmethane | | | |
| Fragrance | Red Age NA HE029-13 | 0.40 | Fragrance | |
| Xantham | Xanthan Gum | 0.30 | Thickener | C |
| Gum | | | | |
| Glycerol | Glycerol | 5.00 | Humectant | |
| EDTA | Tetrasodium EDTA | 0.10 | Chelating | |
| | (ethylenediaminetetraacetic | | agent | |
| | acid | | | |
| | tetrasodium salt) | | | |
| Demineralised | Demineralised water | 43.35 | | |
| water | | | | |

TABLE 2.8
Exemplary composition of the embodiment according to the Example VIII.

| | | Amount | | |
|---|---|---|---|---|
| Ingredient Name | INCI Name | (wt %) | Function | Phase |
| Sorbitan Laurate | Sorbitan Laurate | 3.00 | Emulsifier | A |
| Polyglyceryl-4 | Polyglyceryl-4 | 2.50 | Emulsifier | |
| Laurate/Succinate | Laurate/Succinate | | | |
| (and) Aqua | (and) Aqua | | | |
| Caprylic/capric | Caprylic/capric | 7.34 | Emollient | |
| triglyceride | triglyceride | | | |
| Cocoglycerides | Cocoglycerides | 2.00 | Emollient | |
| Decyl Isosostearate | Decyl Isostearate | 4.00 | Emollient | |
| (and) Isostearyl | (and) Isostearyl | | | |
| Isostearate | Isostearate | | | |
| Polyhydroxystearic | Polyhydroxystearic | 0.66 | Dispersant | |
| Acid | Acid | | | |
| MicNo | Zinc oxide | 12.00 | UV filter | B |
| Demineralised | Demineralised water | 62.80 | | C |
| water | | | | |
| Glycerine | Glycerine | 3.00 | Humectant | |
| | Magnesium | 1.00 | Thickener | |
| | Aluminium Silicate | | | |
| Xanthan Gum | Xanthan Gum | 0.50 | Thickener | |
| | Caprylhydroxamic | 1.20 | Preservative | D |
| | Acid (and) Glyceryl | | blend | |
| | Caprylate (and) | | | |
| | Glycerin | | | |

TABLE 2.9
Exemplary composition of the embodiment according to the Example IX.

| Ingredient | | Amount | | |
|---|---|---|---|---|
| Name | INCI Name | (wt %) | Function | Phase |
| | Steareth-2 | 7.0 | Emulsifier | A |
| | Steareth-21 | 3.0 | Emulsifier | |
| | Cetearyl Alcohol | 1.0 | Thickener | |
| | Ethylhexyl Benzoate | 8.0 | Emollient/ | |
| | | | solvent | |
| | Triethylhexanoin | 5.0 | Emollient/ | |
| | | | solvent | |
| | Ethylhexyl | 2.0 | Photostabiliser | |
| | Methoxycrylene | | | |
| | Octocrylene | 5.0 | UVB filter | |
| | Homosalate | 5.0 | UVB filter | |
| | Butyl | 3.0 | UVA filter | |
| | Methoxydibenzoylmethane | | | |
| | Ethylhexyl | 7.5 | UVB filter | |
| | Methoxycinnamate | | | |
| | Polyhydroxystearic Acid | 0.4 | Dispersant | |
| MicNo | Zinc oxide | 8.0 | UVB/UVA | B |
| | | | filter | |
| | Aqua | 42.6 | | C |
| | Propylene Glycol | 4.0 | Humectant | |
| Magnesium | Magnesium Aluminium | 0.4 | Thickener | |
| Aluminium | Silicate | | | |
| Silicate | | | | |
| Xanthan | Xanthan Gum | 0.1 | Thickener | |
| Gum | | | | |
| | Aqua (and) Hydrolyzed | 2.0 | Film former | D |
| | Wheat Protein/PVP | | | |
| | Crosspolymer | | | |
| | Phenoxyethanol (and) | 1.0 | Preservative | |
| | Ethylhexylglycerin | | | |

TABLE 2.10
Exemplary composition of the embodiment according to the Example X.

| Ingredient | | Amount | | |
|---|---|---|---|---|
| Name | INCI Name | (wt %) | Function | Phase |
| | Polyglyceryl-3 | 3.00 | Emulsifier | A |
| | Polyricinoleate | | | |
| | Caprylic-Capric | 21.00 | Emollient/ | |
| | Triglyceride | | carrier | |
| | Pentaerithrityl | 8.00 | Emollient | |
| | Tetracaprylate/caprate | | | |
| | Jojoba oil | 4.00 | Emollient | |
| | Caprylic/Capric | 8.00 | Inorganic | B |
| | Triglyceride (and) | | UV filter | |
| | Titanium Dioxide (and) | | | |
| | Polyhydroxystearic | | | |
| | Acid (and) Aluminum | | | |
| | Stearate (and) Alumina | | | |
| | Polyhydroxystearic | 0.40 | Dispersant | |
| | Acid | | | |
| MicNo | zinc oxide | 16.00 | Inorganic | |
| | | | UV filter | |
| | Demineralised water | 33.70 | | C |
| | Magnesium Sulphate | 0.70 | Stabilizer | |
| | Heptahydrate | | | |
| | Glycerin | 4.00 | Humectant | |
| | Caprylhydroxamic | 1.20 | Preservative | D |
| | Acid (and) Glyceryl | | blend | |
| | Caprylate (and) | | | |
| | Glycerin | | | |

TABLE 2.11
Exemplary composition of the embodiment according to the Example XI.

| Ingredient | | Amount | | |
|---|---|---|---|---|
| Name | INCI Name | (wt %) | Function | Phase |
| | Sorbitan Laurate | 3.00 | Emulsifier | A |
| | Polyglyceryl-4 | 2.50 | Emulsifier | |
| | Laurate/Succinate | | | |
| | (and) Aqua | | | |
| | Caprylic/capric triglyceride | 14.00 | Emollient | |
| | Squalane | 3.00 | Emollient | |
| | Isostearyl Isostearate | 3.00 | Emollient | |
| | Polyhydroxystearic Acid | 0.84 | Dispersant | |
| MicNo | zinc oxide | 15.00 | UV filter | B |
| | Demineralised water | 51.70 | | C |
| | Glycerine | 3.00 | Humectant | |
| | Magnesium Aluminium | 1.00 | Thickener | |
| | Silicate | | | |
| | Xanthan Gum | 0.50 | Thickener | |
| | Iron Oxides (C.I. 77492) | 0.96 | Yellow | D |
| | (and) Jojoba esters | | pigment | |
| | Iron Oxides (C.I. 77491) | 0.24 | Red | |
| | (and) Jojoba esters | | pigment | |
| | Iron Oxides (C.I. 77499) | 0.06 | Black | |
| | (and) Jojoba esters | | pigment | |
| | Caprylhydroxamic Acid | 1.70 | Preservative | E |
| | (and) Glyceryl Caprylate | | blend | |
| | (and) Glycerin | | | |

What is claimed is:

1. A skincare formulation, comprising polygonal Zinc oxide prismatic platelets composed of primary nanoparticles as a first active substance, wherein the skincare formulation is preservative-free or comprises one or more preservatives in a total concentration of 2% (w/w) or less with respect to a total weight of the skincare formulation.

2. The skincare formulation according to claim 1, comprising the one or more preservatives in a total concentration of 1% (w/w) or less with respect to the total weight of the skincare formulation; or the skincare formulation is preservative-free.

3. The skincare formulation according to claim 1, wherein a prismatic UV filter comprises the polygonal Zinc oxide prismatic platelets composed of the primary nanoparticles, the polygonal Zinc oxide prismatic platelets have a size in a micron-scale and a median specific surface area of more than 25 square meters per gram.

4. The skincare formulation according to claim 3, wherein the polygonal Zinc oxide prismatic platelets comprise the primary nanoparticles in a height direction perpendicular to a plurality of parallel prism base surfaces having an area greater than any prism side surfaces of the polygonal Zinc oxide prismatic platelets, and
the polygonal Zinc oxide prismatic platelets have a thickness providing a light transmittance of 30% or more at a wavelength of 600 nanometers.

5. The skincare formulation according to claim 3, comprising an organic UV filter and/or an inorganic UV filter as a second active substance.

6. The skincare formulation according to claim 5, comprising the organic UV filter as the second active substance, and a concentration of the prismatic UV filter has a concentration of 15 wt. % or lower with regard to the total weight of the skincare formulation.

7. The skincare formulation according to claim 3, wherein the polygonal Zinc oxide prismatic platelets comprise silver as a metal in an elementary form, or in a form of one or more oxides and/or one or more salts of the one or more oxides.

8. A sunscreen, comprising the skincare formulation according to claim 1.

9. A rash crème, comprising the skincare formulation according to claim 1.

10. A moisturizing crème, comprising the skincare formulation according to claim 1.

11. A hair gel, comprising the skincare formulation according to claim 1.

12. A lipstick or lip moisturizing stick, comprising the skincare formulation according to claim 1.

13. A lip balm, comprising the skincare formulation according to claim 1.

14. A hand sanitizer, comprising the skincare formulation according to claim 1.

15. A method of using polygonal Zinc oxide prismatic platelets composed of primary nanoparticles, in a skincare formulation, comprising using the polygonal Zinc oxide prismatic platelets as a preservative for increasing a shelf-life.

16. The skincare formulation according to claim 2, wherein a prismatic UV filter comprises the polygonal Zinc oxide prismatic platelets composed of the primary nanoparticles, the polygonal Zinc oxide prismatic platelets have a size in a micron-scale and a median specific surface area of more than 25 square meters per gram.

17. The skincare formulation according to claim 4, comprising an organic UV filter and/or an inorganic UV filter as a second active substance.

18. The skincare formulation according to claim 4, wherein the polygonal Zinc oxide prismatic platelets comprise silver as a metal in an elementary form, or in a form of one or more oxides and/or one or more salts of the one or more oxides.

19. The skincare formulation according to claim 5, wherein the polygonal Zinc oxide prismatic platelets comprise silver as a metal in an elementary form, or in a form of one or more oxides and/or one or more salts of the one or more oxides.

20. The skincare formulation according to claim 6, wherein the polygonal Zinc oxide prismatic platelets comprise silver as a metal in an elementary form, or in a form of one or more oxides and/or one or more salts of the one or more oxides.
